(19) United States
(12) Patent Application Publication
(10) Pub. No.: US 2003/0045787 A1
(43) Pub. Date: Mar. 6, 2003

(54) Apparatus and method for recording an electrocardiogram using non-obtrusive sensors

(76) Inventors: Arthur E. Schulze, Wharton, TX (US); Waneta C. Tuttle, Albuquerque, NM (US)

Correspondence Address: ROBERTS ABOKHAIR & MARDULA, SUITE 1000, 11800 SUNRISE VALLEY DRIVE, RESTON, VA, 20191, US

(21) Appl. No.: 10/235,998

(22) Filed: Sep. 5, 2002

Related U.S. Application Data
(60) Provisional application No. 60/317,341, filed on Sep. 5, 2001.

(51) Int. Cl. A61B 5/04

(52) U.S. Cl. 600/382

(57) ABSTRACT

The present invention is to a method and apparatus to allow a portable patient data monitor to obtain an electrocardiogram (ECG) for transmission, such as over the Internet. A small electrical conductive patch is added to a sensor which is currently placed in the ear. This patch is used as a “dry” electrode to sense one side of the electrocardiogram signal generated within the subject. A second electrical conductive patch is added to the enclosure of the patient data monitor or other instrumentation package attached or located in an area near the waist of the patient. Wires or wireless interconnections are used to connect these two patches to an electrocardiogram amplifier.

RELATIONSHIP TO OTHER APPLICATIONS

This application claims priority to U.S. Provisional Application Ser. No. 60/371,341, filed Sep. 5, 2001, hereby incorporated by reference.

### BACKGROUND OF THE INVENTION

This invention relates generally to data acquisition and communication devices using sensors in contact with the human body. More particularly, the present invention is a system and method for acquiring, processing, and communicating an electrocardiogram using a multi-variable sensor in the ear of the patient and the case of a data acquisition unit which is used to transmit the data in a wireless mode over the Internet.

The present invention is usable with and related to the co-inventor's U.S. Pat. Nos. 5,673,692 and 6,443,890 and portions of the co-pending and commonly owned U.S. patent application Ser. Nos. 09/783,913 and 09/860,950, hereby incorporated by reference, as applied to generalized acquisition and transfer of data to central Internet databases. The invention allows the transduction of a clinically relevant lead of the electrocardiogram using the location of the multi-variable, single-site sensor that was described in U.S. Pat. No. 5,673,692 combined with the modified case the Patient data monitor which has previously been described U.S. Pat. No. 6,443,890 and in the above-mentioned U.S. Patent Applications.

The purpose of this invention is to add the electrocardiogram to the signals currently being acquired in a non-obtrusive manner without requiring additional placement of sensor sites on the patient.

Obviously, the same invention is useful independent of the multi-variable sensor and the wireless communication capabilities of the Patient data monitor.

### BRIEF SUMMARY OF THE INVENTION

The present invention provides for the measurement of an ECG signal between an ear sensor and a waist sensor. In a preferred embodiment, the ear sensor having an additional “dry” electrode sends data to a waist-mounted Patient data monitor that includes a second electrode on a side adjacent the patient.

It is an object of the invention to allow the recording of an electrocardiogram between a sensor located in an ear of the subject and an electrocardiographic amplifier located on or near the waist of the subject.

It is another object of the invention to provide sensors that utilize dry electrode technology and can be applied by the subject without special effort when the instrumentation is placed in the ear and the device is carried on the waist.

It is another object of the invention to enable the recording of a single lead of the electrocardiogram which approximates a Lead II vector and is best used to measure rate and rhythm in the ambulatory subject.

It is another object of the invention to be designed for automatic data collection without direct interaction by the user or patient.

It is another object of the invention to facilitate the wireless transmission of the electrocardiogram from the ambulatory subject.

It is yet another object of the invention to expand the ear site as a multi-variable sensor location.

It is another object of the invention to provide a sensor wiring system that is non-obtrusive, since it appears to be a earpiece and cell phone when in use.

### BRIEF DESCRIPTION OF THE DRAWINGS

FIG. 1 illustrates a typical implementation of the present invention with a wired sensor.

FIG. 2 illustrates a typical implementation of the present invention with a wireless sensor.

FIG. 3 illustrates a prior art modified Lead II vector.

FIG. 4 illustrates a prior art MX Lead vector.

FIG. 5 illustrates the modified vector of the present invention.

### DETAILED DESCRIPTION OF THE INVENTION

U.S. Pat. No. 6,443,890 discloses a single-site multi-variable biosensor for use with a patient data monitor. The present invention is to a method and apparatus to allow such a device to obtain an electrocardiogram (ECG). A small electrical conductive patch is added to a sensor which is currently placed in the ear. This patch is used as a “dry” electrode to sense one side of the electrocardiogram signal generated within the subject. A second electrical conductive patch is added to the enclosure of the patient data monitor (or other instrumentation package) which is usually attached or located in an area near the waist of the subject. Wires or wireless interconnections are used to connect these two patches to an electrocardiogram amplifier.

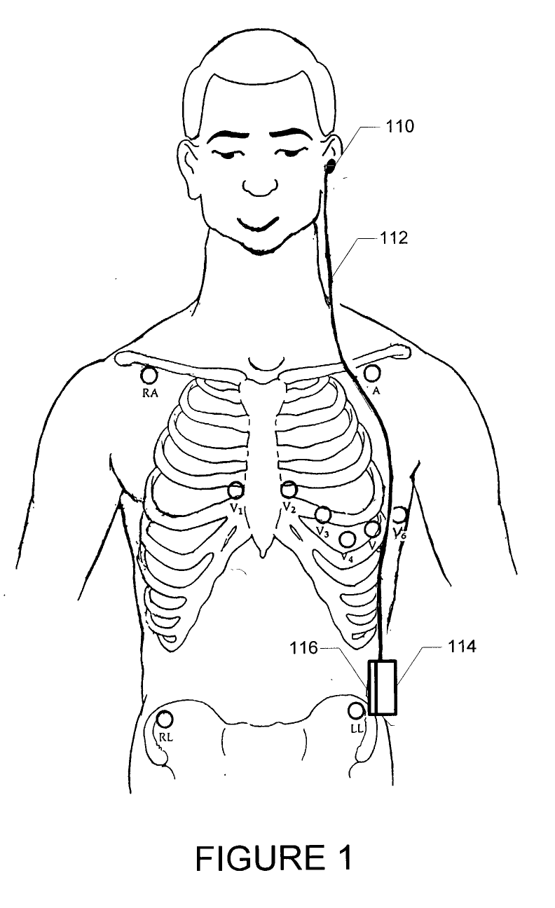

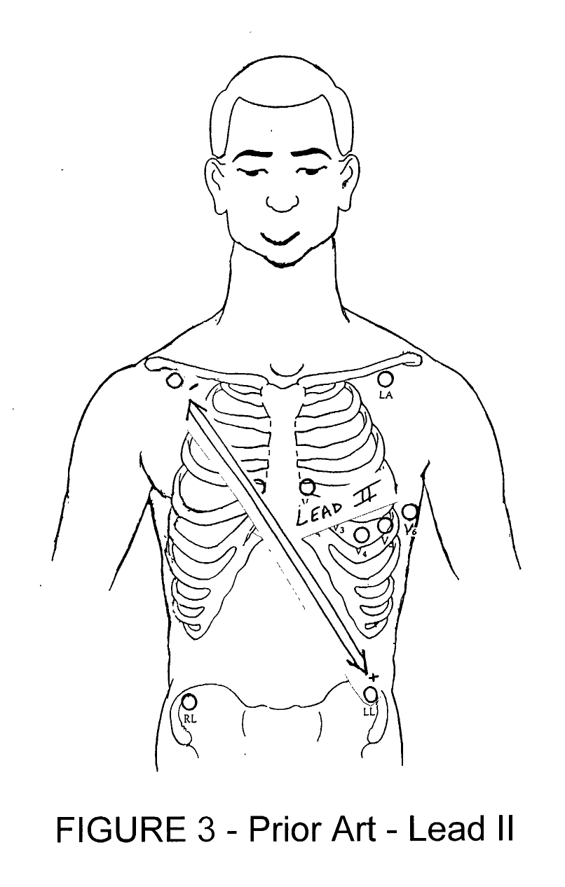

FIG. 1 illustrates the essential features of a first embodiment of the invention. A generally accepted electrocardiographic lead used for monitoring is Lead II, which is a measurement of the electrical potential between the Right Arm and the Left Leg. For monitoring purposes, these locations are usually moved to the RA and LL sites as shown in FIG. 3. Thus, one of the best vectors for use in monitoring the electrocardiogram (ECG) on a continuous basis without encumbering the limbs involves the placement of electrodes near the right shoulder and the below the rib cage on the left side of the subject. These sites can be seen to be electrically equivalent to locations on the right arm and left leg.

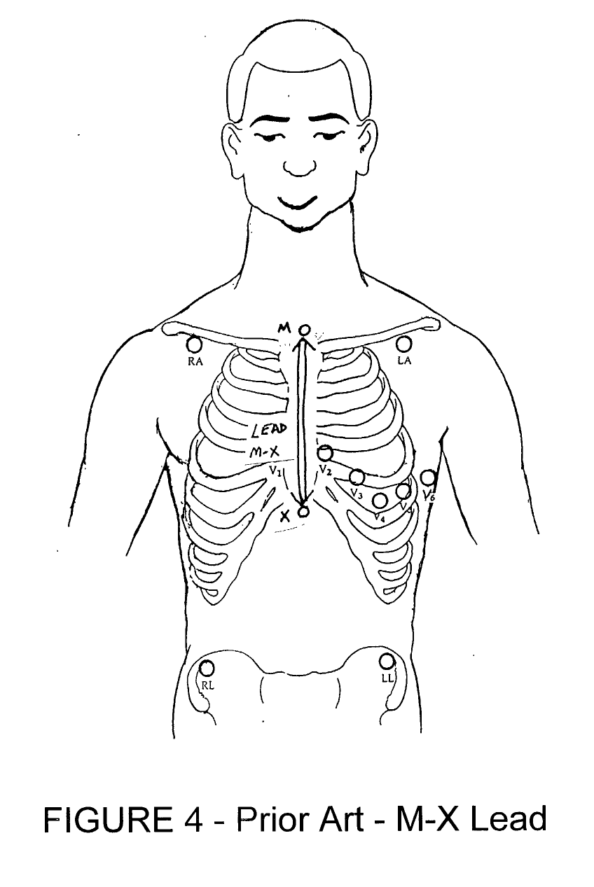

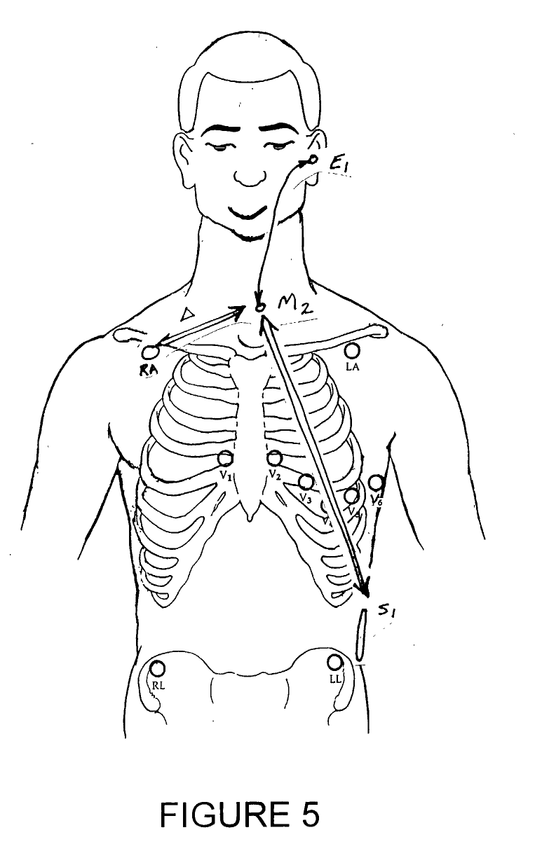

By placing an electrode site on either ear, the electrical equivalent location on the abdomen would be a site located between the RA and LA (near the manubrium). In fact, one of the favored locations for electrodes on the exercising subject (used extensively by the USAF and NASA) is a lead between the manubrium and the xiphoid process (M-X lead), as shown in FIG. 4. Thus, a lead configuration that involves sensors on the ear and on the left waist, below the rib cage would sensor a vector voltage that is substantially similar to the Lead II or M-X leads that are currently being used to monitor the electrocardiogram. The modified vector is as shown in FIG. 5.

As illustrated in FIG. 1, a small electrical conductive patch (not shown) is added to a sensor 110 which is currently placed in the ear. This patch is used as a “dry” electrode to sense one side of the electrocardiogram signal generated within the subject. A second electrical conductive patch 116 is added to the enclosure of the patient data monitor 114 which is usually attached or located in an area near the waist of the subject. A wired interconnection 112 used to connect these two patches to an electrocardiogram amplifier (not shown) in the patient data monitor 114.

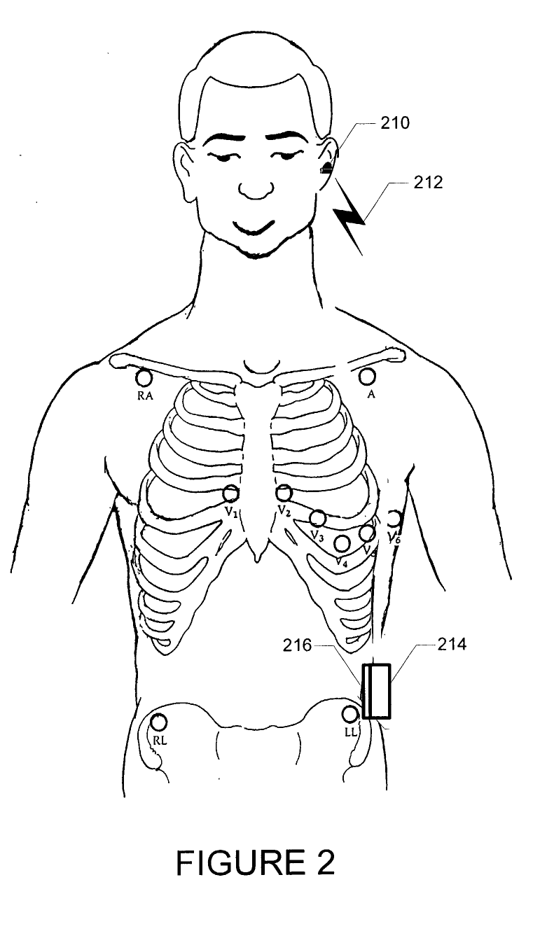

An alternate wireless embodiment is illustrated in FIG. 2, wherein a small electrical conductive patch (not shown) is added to a sensor 210 placed in the ear and wirelessly transmits 212 sensor signals to the patient data monitor 214. A second electrical conductive patch 216 on the enclosure of the patient data monitor 214 is again attached or located in an area near the waist of the subject. A wireless interconnection 212, such as an RF transmitter, is used to connect the ear-emplaced patch to an electrocardiogram amplifier (not shown) in the patient data monitor 214.

The materials that are selected for use to construct the sensors and contact the skin must meet all applicable biocompatibility requirements. Although there are numerous suitable materials, those materials that are generally used in the manufacture of electrocardiographic electrodes are best used in the construction of these sensors. These materials include stainless steel and silver/silver chloride. The electrochemically reversible characteristics of a homogeneous or layered construction using silver/silver chloride has been proven to enhance the signal/noise performance of ECG electrodes. In the case of the multivariable ear sensor, the metal contact material can also serve as shielding material for the case that surrounds the sensor elements. This would improve the overall performance of the sensor and the system in the presence of external noise sources.

The sensor used in the ear can be as small as 10 square millimeters, since it is in a stabilized direct contact location within the ear. The sensor on the patient data monitor should be as large as feasible. If this sensor is worn in direct contact with the skin (under the undergarments), then it can be only slightly larger than less than 100 square millimeters in surface area. If it is worn over a thin undergarment, then it should be as large as possible (5,000 square millimeters or more). When worn over a thin undergarment, a drop of saline solution will help to improve the stability of the signal.

The amplifier used to interface the sensors to the remainder of the system should meet all those requirements generally applied by practitioners of the art of ECG amplifier design. A critical requirement for any amplifier used with this invention is high input impedance and common mode rejection. Analog and digital filtering should be used to enhance the signal-to-noise ratio when used on the ambulatory patient.

Although disclosed with reference to specific examples, one of ordinary skill in the art will recognize numerous modifications can be made without departing from the scope of the invention, which is limited only by the following claims.

We claim:

1 A method for acquiring an electrocardiogram with a portable patient data monitor, comprising:
providing a first electrode patch on a surface of said patient data monitor;
providing a second electrode patch on a surface of an ear-emplaceable sensor;
positioning the first electrode patch adjacent a patient's waist;
positioning the second electrode patch adjacent a patient's ear; and
connecting said first and second electrode patches to an electrocardiogram amplifier to obtain an ECG signal for transmission by said patient data monitor.

2 The method of claim 1, wherein said second patch is connected to said electrocardiogram amplifier using a wire.

3 The method of claim 1, wherein said second patch is connected to said electrocardiogram amplifier using an RF transmitter.

4 The method of claim 1, further comprising providing saline solution to said first electrode patch when positioned adjacent a thin garment.

5 The method of claim 1, further comprising providing analog and digital filtering to said ECG signal.

6 The method of claim 1, wherein said ear-emplaceable sensor includes other sensors and said second electrode patch provides shielding.

7 An apparatus for obtaining an electrocardiogram with a portable patient data monitor, comprising:
a portable patient data monitor including data transmission means, a first electrode patch on an exterior surface thereof, an electrocardiogram amplifier, and a means to position the monitor adjacent a patient's waist;
an ear-emplaceable sensor including a second electrode patch on a surface thereof;
interconnections for each of said first and second electrode patches to said electrocardiogram amplifier.

8 The apparatus of claim 7, wherein said first electrode patch is approximately 100 mm².

9 The apparatus of claim 7, wherein said second electrode patch is approximately 10 mm².

10 The apparatus of claim 7, wherein said first electrode patch is grater than approximately 5000 mm².

11 The apparatus of claim 7, wherein said first and second electrode patches are formed of material selected from stainless steel, homogeneous silver/silver chloride, and layered silver/silver chloride.

12 The apparatus of claim 7, wherein said ear-empaceable sensor includes other sensors and the second electrode patch forms shielding.
